SEQUENCE LISTING (2 sequences)

SEQ ID NO: 1 - DNA, 18 bp, ARTIFICIAL SEQUENCE
  PCR PRIMER
tgtttgcgtctctcccag

SEQ ID NO: 2 - DNA, 18 bp, ARTIFICIAL SEQUENCE
  PCR PRIMER
cacagccctgtgcctctt

(19) United States
(12) Patent Application Publication
(10) Pub. No.: US 2004/0091915 A1
(43) Pub. Date: May 13, 2004

(54) Methods for predicting survival rate in patients having multiple sclerosis

(76) Inventors: David Comings, Monrovia, CA (US); James MacMurray, Claremont, CA (US); Lawrence A. Cone, Palm Springs, CA (US); Radhika Gade-Andavolu, Cathedral City, CA (US)

Correspondence Address: ROTHWELL, FIGG, ERNST & MANBECK, P.C., 1425 K STREET, N.W., SUITE 800, WASHINGTON, DC, 20005, US

(21) Appl. No.: 10/628,554

(22) Filed: Jul. 29, 2003

Related U.S. Application Data
(60) Provisional application No. 60/399,157, filed on Jul. 30, 2002.

(51) Int. Cl. C12Q 1/68

(52) U.S. Cl. 435/6

(57) ABSTRACT

A deletion mutant associated with CCR5, particularly a CCR5 delta 32 deletion mutant, and its use for predicting the survival rate of patients having multiple sclerosis.

This invention claims priority to co-pending U.S. Provisional Application Serial No. 60/399,157 filed Jul. 30, 2002.

### FIELD OF THE INVENTION

This invention relates to genotyping and particularly to the detection of a genetic variant for predicting survival rate or time in patients having multiple sclerosis.

### BACKGROUND OF THE INVENTION

A Multiple Sclerosis (MS) is a chronic demyelinating disorder pathologically characterized by an infiltration of monocytes and T-lymphocytes into the brain parenchyma, destruction of oligodendrocytes and the loss of myelin. The role of chemokines and chemokine receptors is particularly important in MS, in which myelin-destructive inflammation occurs inside the blood-brain barrier and is related to influx of peripheral pro-inflammatory T cells into the CNS. Chemokines play a significant role in the migration of monocytes and T cells and also have been implicated in the onset or progression of MS and experimental autoimmune encephalomyelitis (EAE) (Ransohoff, R. M., et al. 1999; Karpus, W. J. et al., 1995).

Causes of death in MS cases have not been well studied, although the loss of myelin and the subsequent loss of ability to maintain adequate heat reaction and response to infections is likely to play a role. In a study of 6,068 MS cases from the Danish Multiple Sclerosis Registry, MS was noted on the death certificate as the underlying cause of death in 55.4% of cases (Koch-Henriksen N, et al., 1998), but the mechanism by which MS contributes to ‘premature’ death has not been explicated. However, young age at onset and an initial remitting clinical course have both been shown to be significantly associated with longer survival (Midgard R, et al., 1995). To date, studies on survival of MS patients in relation to genotypes have not been specifically addressed.

Chemokine receptor 5 (CCR5), a seven transmembrane-spanning G-protein coupled receptor, is a specific binding site for the CC-chemokines and is expressed on T-helper-1 (TH1) but not on T-helper-2 (TH2) subsets of lymphocytes. Several studies have ascribed to CCR5 surface expression levels an important role in HIV-1 entry and pathogenesis (Huang Y, et al., 1996), and a CCR5Δ32 mutation (homozygous deletion) almost invariably protects from HIV-1 infection. (Biti R., et al., 1997; O'Brien T R et al., 1997). The heterozygotes demonstrate a delay in progression of the disease (deRoda Husman A -M, et al., 1997; Mummidi S., et al., 1998; Daar E S, et al., 1999), lower viral loads and higher CD4+ counts (Cone L. A., et al., 2000). Aberrant production of chemokines has been described in humans and experimental CNS demyelinating lesions (Balashov K E, et al., 1999; Sorensen T L, et al., 1999). Beta-chemokine receptors were examined in post-mortem MS CNS tissue by immuno-histochemistry, and an elevated expression of CCR2, CCR3 and CCR5 was noted (Simpson J., et al., 2000; Zhang G Z, et al., 2000; Strunk T, et al. 2000). This over-expression could be due to a viral infection such as HHV6 that has been proposed to be associated with MS (Challoner P B, et al., 1995; Sanders V J, et al., 1996; Knox K K, et al., 2000).

The ligands for CCR5 include RANTES (regulated-upon-activation, normal T expressed and secreted), macrophage inflammatory protein-1 alpha (MIP-1 alpha) and MIP-1 beta. These ligands belong to the group of CC or beta chemokines and are found principally on TH1 helper cells. (Bonecchi R, et al., 1998). Earlier studies in HIV infected individuals have shown that T-cells from CCR5Δ32 heterozygotes express 5- to 10-fold less CCR5 than wild types after stimulation and, in addition, CD4+ T-cell clones from two homozygous persons for the CCR5Δ32 mutation produced approximately 20-fold more RANTES than wild type clones (Paxton W A, et al. 2001). Analysis of cytokine and cytokine receptor gene expression in MS samples showed predominantly increased levels of several Th1 molecules (TGF-ss, RANTES, and macrophage-inflammatory protein (MIP)-1alpha), although some Th2 genes (IL-3, IL-5, and IL-6/IL-6R) were found to be up-regulated as well (Baranzini S E, et al., 2000).

Genetic studies reveal MS to be polygenic, due to multiple gene associations (Ebers G C, et al., 1994; Oksenberg J R, et al., 1996; Sawcer, S, et al., 1996). Reports of epidemiologic studies of MS (Poser C M, 1994) concluded that the disease was common in persons of Scandinavian descent: an ethnic group that exhibits a high prevalence of CCR5Δ32 mutation. Among the European white population there is a north to south gradient of prevalence of the CCR5Δ32 mutant allele, with the allelic frequencies highest in Scandinavia (16%) and lowest in Sardinia (4%), with a mean allelic frequency across the whole Europe of 9.1% (Martinson, J J, et al., 1997). But the CCR5Δ32 variant was found to be only 1% in individuals of African origin and 9.8% in Caucasians. (Lucotte G, et al., 1998).

CCR5Δ32 is a polymorphism of the CCR5 gene that results in an abnormally truncated CCR5 protein, which is not expressed on the cell surface. With but a few rare exceptions (Dean M, et al., 1996; Wu L, et al., 1997), individuals homozygous for the CCR5Δ32 allele are resistant to HIV-1 infection, while heterozygotes appear to exhibit a slower progression of disease (Biti R, et al., 1997; O'Brien T R, et al. 1997) and more favorable immunologic parameters (Cone, L. A, et al., 2000). Since recent reports imply an association of MS with HHV6 infection, a possible role of CCR5Δ32 mutation and HHV6 in MS was investigated. (Challoner P B, et al., 1995; Sanders V J, et al., 1996; Knox K K, et al., 2000). An open reading frame within HHV-6 designated as U12 gene encodes RANTES (Isegawa Y, et al., 1998), which is a major ligand for HIV-1 co-receptor CCR5 (Deng H, et al., 1996; Dragic T., et al. 1996) and blocks HIV-CCR5 interactions (McDermott D H, et al., 2000; Stantchev, Broder, 2000). In MS, the presence of higher concentrations of RANTES in homozygotes compared to heterozygotes and both compared to wild type CCR5, could predispose someone to more aggressive CNS disease by HHV6 (Milne R S, et al., 2000). Further reports confirm that although HHV6 uses CD46 as a cellular receptor (Santoro F, et al., 1999), HHV6 binds to RANTES, which could modulate a protective inflammatory response and assist in the spread of the virus by immune evasion (Knox K K, et al., 2000).

Previous studies have examined the role of the CCR5 polymorphism in MS (Barcellos et al., 2000). Barcellos et al. showed that the presence of a CCR5Δ32 mutation resulted in a 3.2 year delay in the onset of familial MS, while Sellebjerg et al. (Sellebjerg F, et al., 2000) indicated that the age of onset of disease was somewhat lower (about 3 years) in patients carrying CCR5Δ32. Bennetts' et al. (Bennetts B H, et al., 1997) failed to demonstrate any difference.

Based upon the key role played by chemokines in the migration of macrophages and T cells in MS, the importance of genetic factors associated with, and the possible viral infection involved in, the pathogenesis of the disease, the role of the naturally occurring 32 bp deletion (delta 32 allele) in the CCR5 gene in patients with MS was investigated.

### SUMMARY OF THE INVENTION

In the present invention, an association was found to exist between survival rates of MS patients and the presence of a mutation in the CCR5 gene.

In one embodiment, the invention thus provides a method for predicting the survival rate or time of a subject having multiple sclerosis. The method comprises obtaining a sample from a subject having multiple sclerosis. The DNA of the sample is then evaluated for the presence of a mutation in the CCR5 gene, which mutation correlates to reduced survival of subjects having multiple sclerosis.

The mutation is preferably a deletion, and more preferably CCR5 delta 32.

### BRIEF DESCRIPTION OF THE FIGURES

FIG. 1 is a graph showing the frequency of the CCR5Δ32 mutant allele in MS patients in relation to years of survival (from age of onset to death).

FIG. 2 is a graph showing Kaplan Meier product-limit survival curves for MS patients, separated by CCR5 11 vs. 12 or 22 genotypes.

### DETAILED DESCRIPTION OF THE INVENTION

The presence in MS patients of the CCR5 delta 32 mutation, while not predisposing the patient to developing MS, was concluded to influence the course of the disease.

132 brain samples of deceased MS patients were examined for the CCR5 delta 32 mutation. Results showed that the presence of CCR5 delta 32 made the patients more susceptible to an earlier death. Specifically, observation of the 132 brain samples at autopsy showed that CCR5Δ32 did not protect patients from developing MS when compared to controls, but contrary to expectations, made the MS patients more vulnerable to accelerated disease and earlier death.

DNA was isolated from post mortem brain tissue samples of 132 non-Hispanic Caucasians with MS and from whole blood of 163 healthy subjects. Both groups were screened for the normal 1 allele and for the CCR5Δ32 deletion 2 allele, using the polymerase chain reaction (PCR). Alleles (1 and 2) and genotypes (11, 12 and 22) were counted and their distributions between groups determined. Survival analyses were used to test the effect of CCR5Δ32 deletion on the survivorship.

A significant association of the CCR5 mutant allele was found with early death and with a progressive reduction in the years of survival from age of onset to the age at death with increased frequency of the CCR5 deletion allele (PL≦0.00005). The death hazard ratio of CCR5 12+22 versus 11 genotype is 2.12, after adjusting for the clinical subtype, suggesting MS patients with CCR5 genotype 12 or 22 have twice the mortality as compared to the 11 genotype of patients. Interestingly, this effect is more significant in female MS patients than males, with the hazard ratio of 3.58.

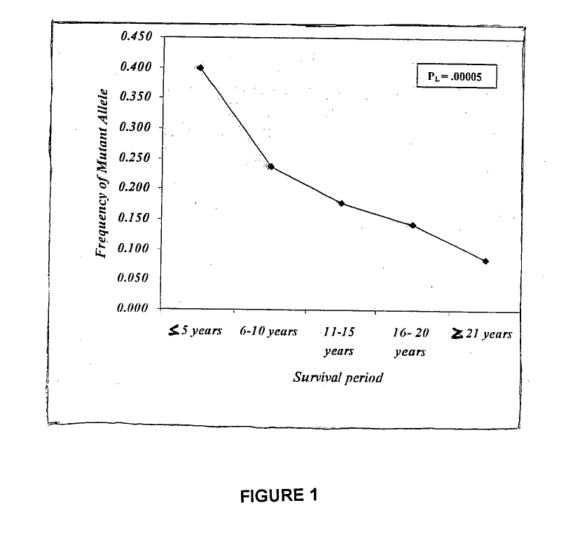

MS subjects and controls were examined for differences in allelic frequencies and genotype prevalence. No significant variation was found among these groups. There was also no significant difference in age at onset of MS (data not shown). However, a marked difference was observed when the Chi-square test was used to examine the potential progressive increase in certain genotypes of MS subjects across five groups with years of survival: ≦5 years, 6-10 years, 11-15 years, 15-20 years and ≧21 years (see Table 1). The MS subjects with 11 genotype survived progressively more years compared to heterozygous subjects who survived progressively fewer years (P<0.00001). Additionally, these groups were examined for possible differences in mutant allelic frequencies by years of survival. A significant association of the CCR5 mutant allele (2) with an early death is shown in FIG. 1 with a progressive reduction in the years of survival from age at onset to the age of death with increased frequency of the CCR5 deletion allele (PL=0.00005).

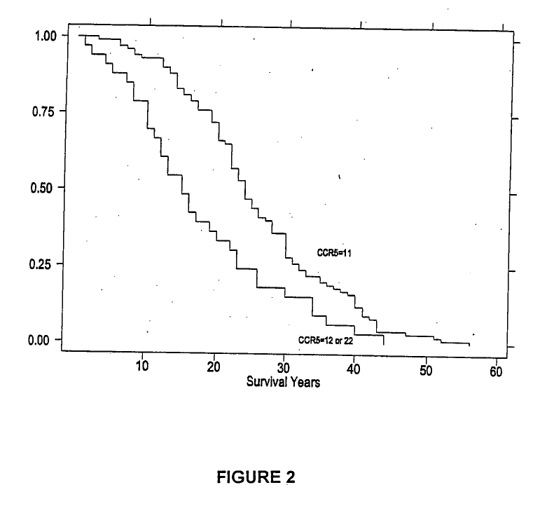

The overall median survival time is 23 years (range 1-56) after the onset of MS. Comparing genotype 11 vs. 12/22 carriers, the median survival times are 24 and 16 years, respectively, while death rates are 0.039 and 0.056 for MS patients. FIG. 2 shows Kaplan-Meier survival curves for patients with CCR5 genotypes. The cumulative death probability is significantly higher in the genotype 12+22 group than in the 11 group (p≦0.00099). For example, the cumulative death probabilities are 34% and 69% for the genotype group 11 and 12+22 after 20 years from onset. The 11 genotype was marginally significantly associated with the more benign clinical subtype (p=0.033, data not shown). Because the survival difference can be confounded by clinical MS subtype and the age of onset, the Cox regression model was used to test the main effect of CCR5 with simultaneous adjustment for these potential effects. The results showed that MS subtype was highly significant (p≦0.0001), while gender and age of onset were not. The hazard ratio was 2.12 for genotype 12+22 versus 11 with p value of ≦0.045 (Table 2A) after adjusting for MS clinical subtype. This means that MS patients with CCR5 genotype 12+22 have over twice the mortality as compared to the 11 genotype, even after adjusting for the subtype. Interestingly, this genotype effect mainly is seen in female patients (Table 2B). The hazard ratio in females was 3.58 (p-value ≦0.001; 95% CI=1.74-7.38). A potential bias may exist because those patients with unknown subtypes may have worse clinical diagnosis and shorter survival years. However, this effect more likely pushes estimates downward (i.e. more conservative) if these patients were associated with CCR5 genotype 12.

The above studies showed a significant gradient in survival for MS patients when individuals having mutant CCR5 genotypes were compared to those with wild type. Thus, the presence of CCR5 mutation in patients with MS showed an exactly opposite effect compared to the protective effect seen in patients with HIV-1 infection (O'Brien T R, et al., 1997; deRoda Husman A -M, et al., 1997).

Finally, the above results allow further speculation that an alteration in the immune response to stimulus (viral perhaps) occurs, whereby the response generated up-regulates RANTES, and the immune response is directed away from the TH1 to TH2 cells. The effects of certain cytokines such as IL-10 and 13 will dominate over IL-2, IL-12 and TNF-alpha. This polarization of T cells also alters their receptor expression and may be precipitated by the CCR5Δ32 mutation.

TABLE 1
Frequency of CCR5Δ32 Genotypes in MS Patients in Relation to Years of
Survival
(Age at Onset to Death)

| | CCR5 Genotypes | | | | | | |
|---|---|---|---|---|---|---|---|
| Years | 11 | | 12 | | 22 | | Total |
| survived | N | (%) | N | (%) | N | (%) | N |
| ≦5 | 2 | 40.0 | 2 | 40.0 | 1 | 20.0 | 5 |
| 6-10 | 8 | 61.5 | 5 | 38.5 | | | 13 |
| 11-15 | 14 | 70.0 | 6 | 30.0 | | | 20 |
| 16-20 | 15 | 75.0 | 5 | 25.0 | | | 20 |
| ≧21 | 67 | 90.5 | 7 | 9.5 | | | 74 |
| Total | 106 | 80.3 | 25 | 18.9 | 1 | .8 | 132 |

| Chi Square | Value | df | Significance |
|---|---|---|---|
| Pearson Chi-Square | 37.260 | 8 | <.00001 |
| Linear-by Linear | 16.463 | 1 | .00005 |
| Association | | | |

TABLE 2A
Effect of CCR5Δ32 on Survivorship in MS patients by Cox
Proportional Hazard Model, with Adjustment of MS subtype
(n = 83)

| | Hazard ratio | | | | |
|---|---|---|---|---|---|
| Factors | (SE) | Z | P-value | Lower 95% | Upper 95% |
| Onset age | 1.03 (0.02) | 2.00 | 0.045 | 1.00 | 1.06 |
| CCR5 | 2.12 (0.56) | 2.74 | 0.006 | 1.24 | 3.62 |
| MS subtype | 5.35 (1.44) | 6.25 | 0.0001 | 3.16 | 9.06 |

TABLE 2B
Effect of CCR5Δ32 on Survivorship in Female MS
patients by Cox Proportional Hazard Model, with
Adjustment of MS subtype (n = 50)

| | | | | Lower | |
|---|---|---|---|---|---|
| Factors | Hazard ratio (SE) | Z | P-value | 95% | Upper 95% |
| CCR5 | 3.58 (1.32) | 3.45 | 0.001 | 1.74 | 7.38 |
| MS subtype | 19.36 (10.24) | 5.60 | <0.0001 | 6.86 | 54.60 |

The invention is further illustrated by the following examples, which are not intended to be limiting.

### EXAMPLE 1

### Study Material

DNA was isolated from post-mortem brain tissue from 132 MS cases from the Human Brain and Spinal Fluid Resource Center at VA Los Angeles Health Care Center, Los Angeles, Calif. All cases were necropsy-confirmed on the basis of white matter lesions and demyelination. The sample was comprised of 47 male and 85 female non-Hispanic Caucasians. The age distribution of MS cases had a range of 30-86, with a mean of 57.2; the age at onset of MS ranged from 18-57, with a mean onset age of 34.6. MS subtyping was obtained by chart review for 83 of the subjects, while the remaining charts provided insufficient data for accurate subtyping. Additionally, because a majority of the progressive forms are preceded by several years of relapsing-remitting course, and because the chart reviews relied more upon recent diagnostic information, the MS subtyping is probably biased toward more progressive forms. The control sample consisted of 163 adult college students from a nearby university (78 males and 85 females), age range 18-49, mean age 34.3 years, from whom blood samples were obtained for genetic studies.

### EXAMPLE 2

### Genetic Analysis

To detect CCR5 delta 32 mutations, genomic DNA was extracted from brain samples and whole blood (Example 1) by standard procedures. A PCR based assay was used to determine the presence of the CCR5 delta 32 deletion. The following oligonucleotides were designed to yield a 232 bp product for the wild type:

Forward—GRL012A* 5′-TGTTTGCGTCTCTCCCAG-3′ and

Reverse—GRL012B* 5′-CACAGCCCTGTGCCTCTT-3′

PCR was performed using an initial denaturation step at 94° C. for 4 minutes, followed by a second denaturation step at 94° C. for 45 minutes, an annealing step at 55° C. for 45 minutes, an elongation step at 72° C. for 45 minutes for 29 cycles, and a final elongation step at 72° C. for 6 minutes. PCR products were run on 12% polyacrylamide gels at constant 150 V for 2 hours and stained with ethidium bromide and viewed on UV. The PCR product obtained had 232 bp for the wild type and a 32 bp deletion leading to 200 bp for the mutant allele. The data obtained on CCR5 delta 32 genotypes were subjected to statistical analysis.

### EXAMPLE 3

### Statistical Analysis

Alleles and genotypes were counted and their distributions between groups were determined. The Chi-square test was employed to statistically compare these groups. All statistical data calculations were done with the SPSS statistical package for Macintosh (release 6.1.1) (SPSS, Inc.; Chicago, Ill.). Survival analysis was applied to compare the survival time after MS onset between the CCR5 genotypes. To estimate the survivor function, Kaplan-Meier product-limit was used (Kaplan E L, et al., 1958). Log-rank test was used to examine the differences between two survivor curves of two CCR5 genotypes (11 versus 12+22). The Cox proportional hazard model (Cox D R, et al., 1984) was also applied to test the multivariate effects including CCR5 genotypic effect, gender and MS age of onset. These analyses were done in STATA 7.0 (Stata Corp., College Station, Tex.).

The above studies thus show a correlation between the CCR5 delta 32 deletion and early death by MS.

In accordance with the present invention, the CCR5 delta 32 deletion may serve as a prognostic marker for MS.

The publications and other materials used herein to illuminate the background of the invention, and to provide additional details respecting the practice of the invention, are incorporated herein by reference as if each was individually incorporated herein by reference.

While the invention has been disclosed by reference to the details of preferred embodiments of the invention, it is to be understood that the disclosure is intended in an illustrative rather than a limiting sense, as it is contemplated that modifications will readily occur to those skilled in the art, within the spirit of the invention and the scope of the appended claims.

### References

1. Karpus, W. J. et al. An important role of the chemokine macrophage inflammatory protein-1 alpha in the pathogenesis of the T cell-mediated autoimmune disease experimental autoimmune encephalomyelitis. J immunol 1995; 155: 5003-10.

2. Ransohoff, R. M. Mechanisms of inflammation in MS tissue: adhesion molecules and chemokines. J Neuroimmunol, 1999; 98: 57-68.

3. Huang Y, Paxton W A, Wolinsky S M, Neumann A, Zhang L, He T et al. The role of a mutant CCR5 allele in HIV-1 transmission and disease progression. Nature Med 1996; 2:1240-3.

4. Biti R, French R, Young J, Bennetts B, Stewart G. HIV infection in an individual homozygous for the CCR5 deletion allele. Nature Med 1997; 3:252-3.

5. O'Brien T R, Winkler C, Dean M, Nelson J A E, Carrington M, Michael N L et al. HIV-1 infection in an homozygous for CCR5 delta 32. Lancet 1997; 349:1219-20.

6. deRoda Husman A -M, Koot M, Cornelissen M, Keet IPM, Brouwer M, Broersen S M, et al. Association between CCR5 genotype and the clinical course of HIV-1 infection. Ann. Intern Med 1997; 127:882-90.

7. Mummidi S, Ahuja S, Gonzalez E, Anderson S A, Santiago E N, Stephan K T, et al. Genealogy of the CCR5 locus and chemokine system gene variants associated with altered rates of HIV-1 disease progression. Nature Med 1998; 4:786-93.

8. Daar E S, Lynn H, Donfeld S, Gompers E, Hilgartner M W, Hoots K, et al. Effects of plasma HIV RNA, CD4+ lymphocytes and the chemokine receptors CCR5 and CCR2b on HIV disease progression in hemophiliacs. JAIDS 1999; 21: 317-25.

9. Cone L. A, Espinoza G, Moreno A, Downs M. CC-CKR5 (CCR5) delta 32 alters the clinical course and immune status of HIV infection. 13^{th} Int. AIDS conference, Durban, South Africa, Jul. 8-13, 2000; TuPeA 3107, p 320.

10. Balashov K E, Rottman J B, Weiner H L, Hancock W W. CCR5 (+) and CXCR3 (+) T cells are increased in multiple sclerosis and their ligands MIP-1alpha and IP-10 are expressed in demyelinating brain lesions. Proc Natl Acad Sci USA 1999; 96: 6873-8.

11. Sorensen T L, Tani M, Jensen J, Pierce V, Lucchinetti C, Folcik V A, et al. Expression of specific chemokines and chemokine receptors in the central nervous system of multiple sclerosis patients. J Clin Invest 1999; 103: 807-15.

12. Simpson J, Rezaie P, Newcombe J, Cuzner M L, Male D, Woodroofe M N. Expression of the beta-chemokine receptors CCR2, CCR3 and CCR5 in multiple sclerosis central nervous system tissue. J Neuroimmunol 2000; 108: 192-200.

13. Zhang G X, Baker C M, Kolson D L, Rostomi A M. Chemokines and chemokine receptors in the pathogenesis of multiple sclerosis. Mult Scler 2000, 6: 3-13.

14. Strunk T, Bubel S, Mascher B, Schlenke P, Kirchner H, Wandinger K P. Increased numbers of CCR5+ interferon-gamma- and tumor necrosis factor-alpha-secreting T lymphocytes in multiple sclerosis patients. Ann Neurol 2000; 47: 269-73.

15. Challoner P B, Smith K T, Parker J D, MacLeod D L, Coulter S N, Rose T M et al. Plaque-associated expression of human herpesvirus 6 in multiple sclerosis. Proc Natl Acad Sci USA 1995; 92: 7440-4.

16. Sanders V J, Felisan S, Waddell A, Tourtellotte W W: Detection of herpesviridae in postmortem multiple sclerosis brain tissue and controls by polymerase chain reaction. J Neurovirol August 1996; 2(4): 249-58.

17. Knox K K, Brewer J H, Henry J M, Harington D J, Carrigan D R. Human herpesvirus 6 and multiple sclerosis. Systemic active infections in patients with early disease. Clin Infect Dis 2000; 31: 894-903.

18. Bonecchi R, Biranchi G, Bordignon P P, D'Aambrosio D, Lang R, Borsatti A et al. Differential expression of chemokine receptors and chemotactic responsiveness of type1 T helper cells (TH1s) and TH2s. J Exp Med 1998; 187: 129-34.

19. Paxton W A, Neumann A U, Kang S, Deutch L, Brown R C, Koup R A, Wolinsky S M. RANTES production from CD4+ lymphocytes correlates with host genotype and rates of human immunodeficiency virus type 1 disease progression. J Infect Dis Jun. 1, 2001; 183 (11): 1678-81.

20. Baranzini S E, Elfstrom C, Chang S Y, Butunoi C, Murray R, Higuchi R, Oksenberg J R; Transcriptional analysis of multiple sclerosis brain lesions reveals a complex pattern of cytokine expression J Immunol, Dec. 1, 2000; 165(11): 6576-82.

21. Ebers G C, Sadovnick A D. The role of genetic factors in multiple sclerosis susceptibility. J. Neuroimmunol 1994; 54: 1-17.

22. Oksenberg J R, seboun E, Hauser S L. Genetics of demyelinating diseases. Brain Pathol 1996; 6: 289-302.

23. Sawcer S, Jones H B, Feakes R, Gray J, Smaldon N, Chatway J, et al. A genome screen in multiple sclerosis reveals susceptibility loci on chromosome 6p21 and 17q22. Nature Genet 1996; 13: 464-8.

24. Poser C M. The dissemination of multiple sclerosis: a Viking saga? A historical assay. Ann Neurol, 1994; 36(S2): S231-43.

25. Martinson J J, Chapman N H, Rees D C, Liu Y. T, Clegg J B. Global distribution of the CCR5 gene 32 base pair deletion. Nature Genet, 1997; 16: 100-2.

26. Lucotte G, Mercier G. Distribution of the CCR5 gene 32-bp deletion in Europe. J Acquire immune def syn Hum Retro virol 1998; 19:174-7.

27. Kaplan E L and Meier P. Nonparametric estimation from incomplete observations. J Am Stat Assoc 53: 457-481, 1958.

28. Cox D R and Oaks D. Analysis of survival data. London UK: Chapman & Hall 1984.

29. Dean M, Carrington M, Winkler C, Huttley G A, Smith M W, Allikmets, et al. Genetic restriction of HIV-1 infection and progression of AIDS by a deletion allele of the CCR5 structural gene. Hemophilia growth and Development study. Multicenter AIDS Cohort study, Multicenter Hemophilia study, San Franscisco Cohort study, Alive study. Science 1996; 273:1856-62.

30. Wu L, Paxton W A, Kassam N, Ruffing N, Rottman J B, Sullivan N. CCR levels and expression pattern correlate with the infectability by macrophage-tropic HIV-1, in vitro. J Exp Med 1997; 185:1681-91.

31. Isegawa Y, Ping Z, Nakano K, Sugimoto N, Yamanishi K. Human herpesvirus 6 open reading frame U12 encodes a funcitional beta-chemokine receptor. J Virol 1998; 72: 6104-12.

32. Deng H, Liu R, Ellmeier W, Choe S, Unutmaz D, Burkhart M, et al. Identification of a major co-receptor for primary isolates of HIV-1. Nature 1996; 381: 661-6.

33. Dragic T, Litwin V, Allaway G P, Martin S R, Huang Y, Nagashima K A, et al. HIV-1 entry into CD4+ cells is mediated by the chemokine receptor CC-CKR-5. Nature 1996; 381: 667-73.

34. McDermott D H, Beecroft M J, Kleeberger C A, Al-Sharif F M, Ollier W B A, Leitman S F, Detels R, Hajeer A H, Murphy P M: Chemokine RANTES promoter polymorphism affects risk of both HIV infection and disease progression in the Multi Center AIDS Cohort Study. AIDS Dec. 1, 2000; 14 (17): 2671-8.

35. Stantchev, Broder C C T S Consistent and significant inhibition of human immunodeficiency virus type 1 envelope-mediated membrane fusion by beta-chemokines (RANTES) in primary human macrophages. J infect Dis July 2000; 182 (1): 68-78.

36. Koch-Henriksen N, Bronnum-Hansen H, Stenager E. Underlying cause of death in Danish patients with multiple sclerosis: results from the Danish Multiple Sclerosis Registry. J Neurol Neurosurg Psychiatry July 1998; 65(1):56-9.

37. Midgard R, Albrektsen G, Riise T, Kvale G, Nyland H. Prognostic factors for survival in multiple sclerosis: a longitudinal, population based study in More and Romsdal, Norway. J Neurol Neurosurg Psychiatry April 1995; 58(4):417-21.

38. Barcellos L F, Schito A M, Rimmler J B, Vittinghoff E, Shih A, Lincoln R et al. CC-cheokine receptor 5 polymorphism and age at onset in familial sclerosis. Immunogenet 2000; 51: 281-8.

39. Sellebjerg F, Madsen H O, Jensen C V, Jensen J, Garred P. CCR5 delta 32, matrix metallo-proteinase9 and disease activity in multiple sclerosis. J Neuroimmunol 2000; 102:98-106.

40. Bennetts B H, Teutsch S M, Buhler M M, Heard R N S, Stewart G J. The CCR5 deletion mutation fails to protect against multiple sclerosis. Human Immunol 1997; 58: 52-59.

41. Milne R S, Mattick C, Nicholson L, Devaraj P, Alcami A, Gompels U A. RANTES binding and down-regulation by a novel human herpesvirus-6 beta chemokine receptor. J Immunol 2000; 164: 2396-2404.

42. Santoro F, Kennedy P E, ocatelli G, Malnati M S, Berger E A, Lusso P. CD46 is a cellular receptor for human herpes 6. Cell 1999; 99: 817-27.

What is claimed is:

1. A method for predicting the survival time of a subject having multiple sclerosis, comprising:
a) obtaining a sample from a subject having multiple sclerosis; and
b) evaluating the DNA of the sample for the presence of a mutation in the CCR5 gene, wherein the mutation correlates to reduced survival of subjects having multiple sclerosis.

2. The method of claim 1, wherein the mutation is a deletion.

3. The method of claim 2, wherein the deletion is CCR5 delta 32.

4. The method of claim 1, wherein the sample is whole blood.

5. The method of claim 2, wherein the sample is whole blood.

6. The method of claim 3, wherein the sample is whole blood.
